(19) United States
(12) Patent Application Publication
(10) Pub. No.: US 2023/0270931 A1
(43) Pub. Date: Aug. 31, 2023

(54) SELF-POWERED FLUID WARMING CONTAINER AND FLUID WARMING DEVICE FOR USE IN A STERILE SURGICAL FIELD

(71) Applicant: Thomas Ward, Markleeville, CA (US)

(72) Inventor: Thomas Ward, Markleeville, CA (US)

(21) Appl. No.: 17/681,986

(22) Filed: Feb. 28, 2022

(51) Int. Cl. A61M 3/02

(52) U.S. Cl. CPC A61M 3/0245; A61M 3/0202; A61M 2205/3368; A61M 2205/3633; A61M 2205/8206; A61M 2205/3653; A61M 2209/06

(57) ABSTRACT

Embodiments of a fluid warming container and a fluid warming device are described. The fluid warming container defines an interior volume arranged to receive a liquid. The fluid warming container also includes a heating element, a power source, and a switch configured to selectively control a flow of electrical current from the power source to the heating element to warm the liquid. The fluid warming device includes an elongate housing, a heating element, a power source, and a switch configured to selectively control a flow of electrical current from the power source to the heating element to warm a liquid in contact with the fluid warming device.

### BACKGROUND

The field of the disclosure generally relates to a fluid warming container and a fluid warming device, and more particularly to a self-powered fluid warming container and a self-powered fluid warming device for use in a sterile surgical field.

In the medical industry, there are many postoperative and intraoperative cleansing and treatment options available. Specifically, it can be important, during and immediately following a surgical procedure, to wash surgical wounds, such as to mitigate, reduce, and/or eliminate unwanted postoperative infection at the surgical wound site, as well, among many other reasons, as to remove dead and damaged tissue from the area surrounding the wound.

At least some known options for post- and intraoperative wound cleansing include wound irrigation and intracavity lavage. Generally, surgical wound irrigation involves washing a surgical wound with a liquid, such as saline solution (e.g., “normal saline,” which may include a standardized concentration of sodium chloride and water), to remove blood and other tissue as well as to reduce the possibility of infection at the surgical site. Similarly, intracavity lavage refers to a related technique for washing a body cavity exposed by a surgical procedure. In both cases (wound irrigation as well as lavage), water or saline solution may be used. Likewise, in some instances, medicaments may be included to facilitate treatment.

Currently, the saline or other liquid used in irrigation and lavage procedures is dispensed, at room temperature or cold, into a basin. The basin is in an operating room, which has been sterilized and draped out, and which may generally be referred to as a “sterile surgical field.” The basin may be heated to raise the temperature of the liquid, prior to use, to a temperature at or near human body temperature (e.g., 98.6 degrees Fahrenheit), to promote healing as well as to prevent additional stress on the patient during and following the surgical procedure.

In many cases, a member of the surgical team (e.g., a circulating nurse) may transfer liquid from the basin into a suitable vessel, such as any standard medical container. Although the liquid may be maintained at a suitable temperature within the basin, once the liquid has been removed from the basin, the temperature of the liquid may drop rapidly, which may reduce the efficacy and/or desirability of using the liquid for wound washing. In some cases, if the liquid sits too long (e.g., even as little as just a few minutes), it may be necessary to dispose of the liquid in exchange for a new volume of liquid drawn from the basin.

Some operating centers may additionally, or alternatively, include saline warmers. Generally, these warmers are arranged to hold and pre-warm one or more bottles of saline. Specifically, bottles may be kept in the warming and retrieved, during a surgery, by the circulating nurse for use as desired. Typically, however, the operating center (which may include a plurality of individual operating rooms) is supplied with a single saline warmer, and in any event, it is uncommon that each operating room would be individually outfitted with a respective saline warmer.

As a result, although saline (or other liquid) may be maintained at a suitable temperature within a saline warmer, once the bottle containing the liquid is removed from the warmer, the temperature of the liquid begins to drop, and it may again be necessary to exchange the bottle for a warmer bottle if the liquid in the bottle is not used within a span of just a few minutes. This shortcoming is exacerbated by the fact that individual operating rooms are typically not equipped with warmers of their own, such that the circulating nurse must exit the operating room to retrieve a bottle from the warmer serving the entire surgical center.

A container capable of warming and/or maintaining a liquid, such as saline, at a desired temperature, or in a desired temperature range (such as at or near human body temperature) is therefore desirable. More particularly, a fluid warming container capable of warming and/or keeping liquid warm for the duration of a surgery is desirable. Likewise, a fluid warming device that can be introduced in a standard medical container to warm a liquid therein, such as for the duration of a surgery, is also desirable.

### BRIEF SUMMARY

In one aspect, a fluid warming container is described. The fluid warming container includes a base portion, and a sidewall portion coupled at a first end thereof to the base portion and extending away from the base portion to define an interior volume of the fluid warming container. The interior volume is arranged to receive a liquid. The fluid warming container also includes a heating element coupled to one of i) the base portion or ii) the sidewall portion, a power source, and a switch electrically connected to the power source and the heating element. The switch is configured to selectively control a flow of electrical current from the power source to the heating element, whereby when the heating element receives the flow of electrical current, a temperature of the heating element increases.

In another aspect, a fluid warming device is described. The fluid warming device includes an elongate housing having a first end, a second end, and a body extending therebetween. The fluid warming device also includes a heating element coupled to the housing, a power source disposed within the housing, and a switch electrically connected to the power source and the heating element. The switch is configured to selectively control a flow of electrical current from the power source to the heating element, whereby when the heating element receives the flow of electrical current, a temperature of the heating element increases.

In yet another aspect, a sterile packaged medical device is described. The sterile packaged medical device includes a package including a casing. The casing defines a sterile interior region. The sterile packaged medical device also includes one of: a fluid warming container enclosed within the sterile interior region of the casing, or a fluid warming device. The fluid warming container, if included in the package, includes a base portion, a sidewall portion coupled at a first end thereof to the base portion to define an interior volume of the fluid warming container. The interior volume is arranged to receive a fluid. The fluid warming container also includes a heating element, a power source, and a switch electrically connected to the power source and the heating element. The switch is configured to selectively control a flow of electrical current from the power source to the heating element, whereby when the heating element receives the flow of electrical current, a temperature of the heating element increases. The fluid warming device, if included in the package, includes a housing having a first end, a second end, and a body extending therebetween. The fluid warming device also includes a heating element, a power source disposed within the housing, and a switch electrically connected to the power source and the heating element. The switch is configured to selectively control a flow of electrical current from the power source to the heating element, whereby when the heating element receives the flow of electrical current, a temperature of the heating element increases.

### BRIEF DESCRIPTION OF THE DRAWINGS

FIG. 1 is a perspective view of an example embodiment of a fluid warming container.

FIG. 2 is a cross-sectional view of the fluid warming container shown in FIG. 1 taken along section 2-2 of FIG. 1.

FIG. 3 is a perspective view of an example embodiment of a package containing the fluid warming container shown in FIG. 1, in which a portion of the package is cutaway to show the fluid warming container inside the package.

FIG. 4 is a perspective view of an example embodiment of a fluid warming device.

FIG. 5 is a cross-sectional view of the fluid warming device shown in FIG. 4 taken along section 5-5 of FIG. 4.

FIG. 6 is a perspective view of an alternate example embodiment of a fluid warming device.

FIG. 7 is a cross-sectional view of the fluid warming device shown in FIG. 6 taken along section 7-7 of FIG. 6.

FIG. 8 is a perspective view of an example embodiment of a medical container that includes the fluid warming device shown in FIGS. 4 and 5 during use.

FIG. 9 is a perspective view of an example embodiment of a package containing the fluid warming device shown in FIGS. 4 and 5, in which a portion of the package is cutaway to show the fluid warming device inside the package.

FIG. 10 is a block diagram of an example embodiment of an electrical system for use with the fluid warming container shown in FIGS. 1 and 2 and/or the fluid warming devices shown in FIG. 4-7.

FIG. 11 is a circuit diagram of the electrical system shown in FIG. 10.

FIG. 12 is a block diagram of an alternate example embodiment of an electrical system for use with the fluid warming container shown in FIGS. 1 and 2 and/or the fluid warming devices shown in FIG. 4-7.

FIG. 13 is a flowchart illustrating an example process for manufacturing the fluid warming container shown in FIGS. 1 and 2 and/or the fluid warming devices shown in FIG. 4-7.

FIG. 14 is a flowchart illustrating an example process for packaging the fluid warming container shown in FIGS. 1 and 2 and/or the fluid warming devices shown in FIG. 4-7 in a sterile package.

### DETAILED DESCRIPTION

Embodiments of self-powered (e.g., battery-powered) fluid warming container and fluid warming device are described herein. In the example embodiments, the fluid warming container is arranged to receive and warm a fluid or liquid, such as liquid water, saline, combinations thereof, one or more medicaments and/or combinations of water, saline, and/or medicaments, and/or any other liquid that a user may desire to heat or warm to a particular temperature. Likewise, the fluid warming devices described herein may include a housing that defines elongate member, which is also configured to warm a volume of liquid, such as a volume of liquid contained within a standard medical container, to a desired temperature.

In various embodiments, it may, for example, be desirable to warm the liquid to within a predefined range of a mammalian body temperature prior to performing a surgical procedure on the mammal. In the case of a human patient, it may for instance be desirable to warm the liquid within the fluid warming container to about human body temperature (e.g., 98.6 degrees Fahrenheit). At least one advantage associated with warming the liquid to a temperature at or near human body temperature is, it will be appreciated, to bring the liquid to the same temperature as the patient's body prior to using the liquid during the surgery, where for example, it is common to use the liquid to rinse portions of the patient's anatomy that have been opened and/or exposed by the surgical procedure (e.g., during wound irrigation and/or lavage, as described herein).

Cold or room temperature liquids can be undesirable during surgical procedures for a variety of reasons, including that using liquids that are not sufficiently warm may cause stress on the patient's body (which is already stressed by the surgery itself). Another technical advantage of the present disclosure is thus that the fluid warming container and/or fluid warming devices described herein are useful to alleviate surgical stress on a patient as well as to promote healing. Likewise, embodiments of the present disclosure facilitate the introduction of sterilized and safe liquids during a surgical procedure, which may also be reliably controlled to achieve and maintain a desired temperature close to a mammalian body temperature.

To facilitate a sterile surgical environment (e.g., a sterile “surgical field”), the fluid warming container and/or fluid warming devices described herein may be sterilized or disinfected and/or packaged in sterilized or disinfecting packages. For example, in some embodiments, ethylene oxide may be introduced in a package together with a container and/or device, and the package subsequently sealed, to facilitate and promote sterilization and/or disinfection of the container and/or device inside the package. The container and/or device may be stored inside of the package for any duration of time in the sterile and/or disinfected state until it is desired to unseal the container and/or devices for use during a surgery.

For example, in at least one embodiment, a member of a surgical team may unseal a sterilized and/or disinfected container and/or device by opening the sealed packaging and placing the container or device on a sterile surgical field, such as on a sterile tray or work surface within a draped out and sterilized operating room. Another technical improvement and advantage of the embodiments described herein is thus that medical staff can ensure that the container or device is sterile when it is finally unsealed and placed within the sterile surgical field, since it has remained sealed until that time.

In addition to these features and improvements, the containers and devices described herein may include a standalone energy storage device, such as a rechargeable and/or non-rechargeable battery. The battery may be included in the container and devices during manufacturing, such that the container and devices are self-powered and operable “out of the box” without any necessity of charging the battery prior to use.

Moreover, the batteries used in the various embodiments may be pre-charged to include an electrical charge sufficient at least to warm a specified volume of liquid to a desired temperature (e.g., at or near a mammalian body temperature) and/or, in addition, to maintain the liquid at the desired temperature for the duration of a surgical procedure. The containers and devices described herein may also be used repeatedly during a surgical procedure to warm consecutive volumes of liquid, such as for example, after a preceding volume of liquid is warmed and used as part of the procedure.

In some embodiments, and in addition to the many other advantages described herein, the containers and devices disclosed may be set, such as by a user, to bring liquid to a desired temperature for any desired period of time as well as, in some cases, to prevent overheating the liquid beyond an established maximum temperature. As a result, safety, efficiency, and overall performance are further improved by these and other features.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged. Such ranges are identified and include all the sub-ranges contained therein unless context or language indicates otherwise.

As used herein, the term “sterile surgical field” may refer to any sterile or otherwise disinfected surgical environment, such as an operating room that has been disinfected and/or sterilized and covered or “draped out” with a sterile barrier to create and maintain the sterile surgical field for the purpose of performing a surgical procedure. In some cases, a sterile surgical field may include a variety of work surface, which may also be sterilized such as trays, tables, and the like.

As used herein, spatially relative terms, such as “beneath,” “below,” “under,” “underneath,” “lower,” “higher,” “above,” “over,” “top,” “bottom,” and the like, may be used to describe one element or feature's relationship to one or more other elements or features as illustrated in the figures. It will be understood that such spatially relative terms are intended to encompass different orientations of the elements and features described herein both in operation as well as in addition to the orientations depicted in the figures. For example, if an element or feature in the figures is turned over, elements described as being “below” one or more other elements or features may be regarded as being “above” those elements or features. Thus, exemplary terms such as “below,” “under,” or “beneath” may encompass both an orientation of above and below, depending, for example, upon a relative orientation between such elements or features and one or more other elements or features.

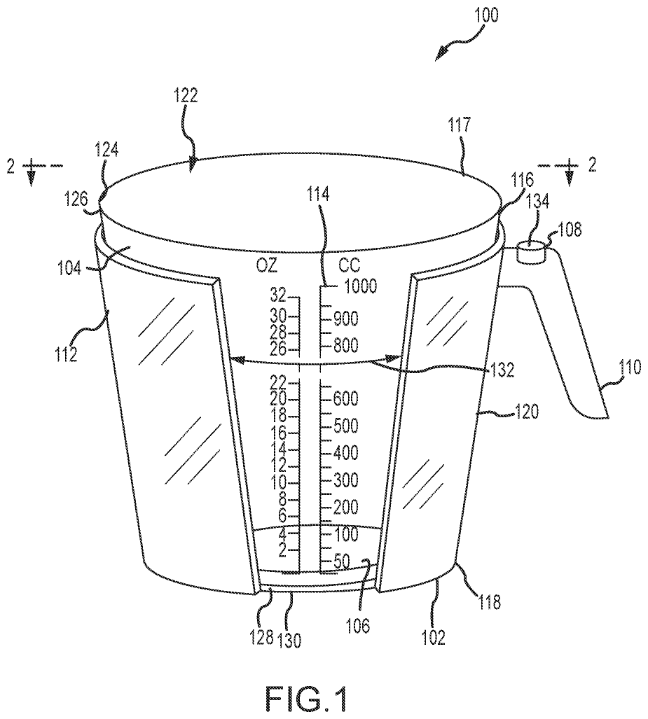

FIG. 1 is a perspective view of an example embodiment of a fluid warming container 100. As described in detail herein, fluid warming container 100 can be used to receive and hold a volume of liquid, such as a volume of water, a volume of saline solution, and the like. During operation, fluid warming container 100 can heat the volume of liquid contained therein to a desired temperature, such as for example, a temperature in a selected or predefined range of human body temperature (e.g., in any desired range of 98.6 degrees Fahrenheit, such as for example, but not limited to, plus or minus 5 degrees Fahrenheit, plus or minus 10 degrees Fahrenheit, etc.) As described herein, fluid warming container 100 may also raise a temperature of liquid to any other desired temperature or range of temperatures.

As a result, fluid warming container 100 can be used during a surgical procedure to bring a volume of liquid contained therein to a desired temperature. Once the liquid has reached the desired temperature, fluid warming container 100 may also maintain the liquid at the desired temperature for the duration of the surgical procedure, or until the liquid is used during the surgery, such as by pouring some amount of the liquid from container 100 onto a surgical site to cleanse a wound and/or into a body cavity to rinse or wash the body cavity.

Accordingly, as shown in the example embodiment of FIG. 1, fluid warming container 100 includes a base portion 102, a sidewall portion 104, a heating element 106, and/or a switch 108. In some embodiments, container 100 also includes a handle 110, although some embodiments may exclude handle 110. In addition, in at least some embodiments, container 100 may include an insulating portion 112 and/or one or more measuring indicia 114.

In at least one embodiment, sidewall portion 104 includes a first end 116, a second end 118, and a body 120 extending therebetween. First end 116 defines an opening 117 for receiving and pouring liquid. First end 116 and second end 118 may include any suitable shape, such as for example, but not limited to, generally circular shapes, oval shapes, triangular shapes, square shapes, and the like. In some embodiments, first end 116 and second end 118 may be substantially congruous (e.g., the same size and/or shape). In other embodiments, first end 116 and second end 118 may be substantially incongruous (e.g., not the same size and/or shape). In some embodiments, first end 116 may also include a spout or mouth to facilitate pouring liquid from container 100.

Accordingly, in various embodiments, sidewall portion 104 may define any of a variety of suitable shapes, dimensions, and/or orientations, such as cylindrical shapes, cup-shapes, and the like. In the example embodiment, sidewall portion 104 defines a cup-shape. However, it will be appreciated that other shapes are within the scope of and contemplated by the present disclosure.

As shown, in the example embodiment, second end 118 of sidewall portion 104 is coupled to and extends away (e.g., substantially orthogonally and/or at any suitable angle) from base portion 102 to define an interior volume 122 of fluid warming container 100, which may as described herein, be arranged to receive and contain a fluid, such as liquid water, saline, compositions and/or mixtures thereof, such as medicated compositions or mixtures thereof, and the like.

In various embodiments, one or more portions of fluid warming container 100 may be composed of any suitable plastic or synthetic material, such as, without limitation, polyethylene terephthalate (PET), high density polyethylene, (HDPE), low density polyethylene (LDPE), polypropylene (PP), and/or any other biologically safe or inert plastic or composition of plastics, such as any plastic suitable for contact with a human body and/or liquids that may be poured from fluid warming container 100 into contact with a human body. In addition, the plastics used may be transparent, translucent, opaque, and/or not transparent or translucent.

In some embodiments, the plastic or plastic composition used in the manufacture of fluid warming container 100 may be heat resistant to a predefined temperature, such that fluid warming container 100 (e.g., base portion 102 and/or sidewall portion 104) do not melt when heating element 106 is active, as described further herein. In some embodiments, the portion of fluid warming container 100 in contact with heating element 106 may be composed of a heat resistant plastic, which may be the same as or different from the plastic used in the construction of portions of fluid warming container 100 that are not in direct contact with heating element 106.

For example, in at least one embodiment, at least the portion of fluid warming container 100 in contact with heating element 106, such as base portion 102 and/or sidewall portion 104, may include any Type 1, 2, 3, 4, 5, 6, and/or 7 plastics, as specified using the US Society of Plastics Industry (SPI) codes, and which are heat resistant, while one or more other portions of fluid warming container 100 may include any other plastic (e.g., Type 1, 2, 3, 4, 5, 6, and/or 7 plastics), which may or may not be less heat resistant, and/or the same plastic. In some embodiments, as described herein, fluid warming container 100 may be manufactured from a single type or composition of plastic, such as entirely from PET, HDPE, LDPE, PP, and the like. In at least one embodiment, heating element 106 may not reach temperatures capable of melting container 100, and container 100 may be made from any biologically suitable plastic.

Moreover, in at least one embodiment, all or a portion of fluid warming container 100 may include a metal composition, such as stainless steel. Stated another way, in at least one embodiment, fluid warming container 100 may be made from stainless steel and/or another suitable metal. Generally, because fluid warming container 100 is intended for use in a surgical setting, glass and other compositions that may shatter or break are not used, although in at least one embodiment, fluid warming container 100 may include glass, such as a shatter-resistant floated borosilicate glass and/or another shatter-resistant glass.

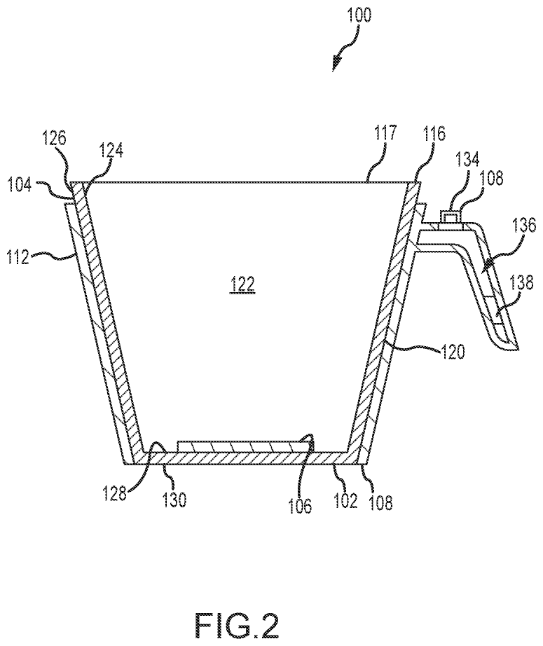

In addition to these features, fluid warming container 100 may include insulating portion 112, as shown, which may in at least some embodiments, be disposed at least partially about sidewall portion 104. More particularly, sidewall portion 104 may include an inner surface 124 and an outer surface 126. As described elsewhere herein and as shown in FIG. 1 and FIG. 2, inner surface 124 may define interior volume 122 in association with base portion 102.

Accordingly, in the example embodiment, insulating portion 112 may be coupled to at least a portion of outer surface 126 to cover at least a portion of outer surface 126. In various embodiments, insulating portion 112 may include any suitable thermal insulating material, such as fiberglass insulating materials, polystyrene insulating materials, cellulose materials, polyisocyanurate insulating materials, polyurethane insulating materials, compositions of insulating foam, various insulating facings and/or coatings, such as spray and/or sputtered coatings of insulation materials, rubber, and/or any other suitable material having thermal insulation properties. In at least one embodiment, insulating portion 112 may, for example, include a removable and/or reusable sleeve (e.g., a removable cellulose or foam sleeve), which may be selectively attached to and/or detached from fluid warming container 100. Insulating portion 112 may, in addition, include any suitable thickness, such as for example, and without limitation, ⅛ inch, ¼ inch, ½ inch, ¾ inch, 1 inch, and the like.

Although in the example embodiment, insulating portion 112 is shown coupled to outer surface 126, in at least one embodiment, insulating portion 112 may be coupled to inner surface 124. In some embodiments, more than one insulating portion 112 may be included, such as a first insulating portion 112 coupled to outer surface 126 and a second insulating portion coupled to inner surface 124. Likewise, in some embodiments, insulating portion 112 may extend over all or a portion of base portion 102. For example, a top surface 128 of base portion 102 may include insulating material. In addition, a bottom surface 130 of base portion 102 may include insulating material.

In the example embodiment, insulating portion 112 is coupled to sidewall 104 to define a gap 132. As shown, one or more measuring indicia 114 may be visible through gap 132. In various embodiments, measuring indicia may specify any suitable volume of liquid, and may be graduated or incremented, such as from a minimum volume of liquid to a maximum volume of liquid. Although in the example embodiment, a single gap 132 is illustrated, in at least some embodiments, more than one gap 132 may be included. For example, in at least one embodiment, a second gap may be included in insulating portion 112 substantially diametrically opposite gap 132. The second gap may, in addition, be substantially congruous with gap 132, such that gap 132 and the second gap are symmetrical about sidewall portion 104. In at least one embodiment, a second set of indicia, similar to or the same as indicia 114, may also be included in the second gap.

In the example embodiment, measuring indicia 114 may include a variety of systems of measurement, such as any of an International System of Units (SI) specifying cubic centimeters (cc), liters (L), milliliters (mL), etc., as well as United States customary scale (specifying ounces or “oz”). It will be appreciated, however, that a single SI scale, a single United States customary scale, and/or any other desired system of measurement (e.g., British imperial) may be included, as well as that any minimum and maximum volume may be specified as desired. As a result, fluid warming container 100 may be sized and shaped to contain any desired volume of liquid, including as shown, a volume of 1000 cc (or 32 oz) as well as any other desired volume. Stated another way, different sizes and shapes of fluid warming container 100 are contemplated by and within the scope of the present disclosure.

In various embodiments, measuring indicia 114 may be printed, stamped, engraved, painted, and/or otherwise attached or included on either or both of inner surface 124 and/or outer surface 126 of sidewall portion 104. In some embodiments, to facilitate viewing a level of liquid within fluid warming container 100, as described herein, sidewall portion 104 may be substantially transparent and/or translucent, such that the level of liquid is visible through sidewall portion 104. However, in at least one embodiment, measuring indicia 114 may be included on inner surface 124, which may facilitate visually determining a level of liquid within fluid warming container 100 even if sidewall portion 104 is not translucent. In one embodiment, to facilitate reading of measuring indicia 114, fluid warming container 100 may include a light or other illumination device in base portion 102 or elsewhere to illuminate sidewall portion 104 and thus measuring indicia 114 from within. At least one technical improvement associated with including measuring indicia 114 on inner surface 124 is that insulating portion 112 may extend all the way around sidewall portion 104, eliminating the need for gap 132, and increasing the total insulation of container 100.

In addition to these features, in at least some embodiments, inner surface 124 may include a polished or reflective coating, to reflect thermal energy (i.e., heat) back into a volume of liquid contained within fluid warming container 100. In at least one embodiment, measuring indicia 114 may be included on reflective surfaces, as described, and/or in association with one or more illumination devices, which may work especially well to make measuring indicia 114 readable when inner surface 124 is also reflective. In some embodiments, to contain heat and conserve electrical energy (e.g., battery power, as described herein) prior to using the liquid during a surgical procedure, a lid or cap, such as a tight-fitting insulated lid or cap, may be attached to first end 116 of fluid warming container 100 after liquid is received within container 100.

As described above, fluid warming container 100 may include handle 110, which may be coupled to sidewall portion 104 as desired and which may be used to grasp fluid warming container 100. In at least some embodiments, switch 108 may be positioned on handle 110, as shown, to facilitate activation of heating element 106, as described in additional detail herein. In the example embodiment, switch 108 is a pushbutton switch that includes a pushbutton actuator 134. However, it will be appreciated that any of a variety of suitable switches may be used, such as to complete an electrical circuit, as described elsewhere herein, between a power source 138 and heating element 106.

In the example embodiment, heating element 106 is coupled to base portion 102 as shown. For example, heating element 106 may include a substantially planar metallic member (e.g., an electrically conductive metal plate). When electrical current flows through heating element 106, as described in additional detail herein, a temperature of heating element 106 may increase to heat a liquid within container 100. In some embodiments, a diameter of heating element 106 is substantially the same as a diameter defined by second end 118 of sidewall portion 104. In some embodiments, a diameter of heating element 106 is less than the diameter defined by second end 118.

In addition, in at least some embodiments, heating element 106 may be coupled to inner surface 124 of sidewall portion 104, such as instead of and/or in addition to being coupled to base portion 102. For example, heating element 106 may be substantially cylindrical or cup shaped to conform to the shape of all or a portion of sidewall portion 104. Other shapes of heating element 106 are also contemplated by and within the scope of the present disclosure.

Moreover, in various embodiments, heating element 106 may include any of a variety of metal conductors in any of a variety of suitable geometries. For example, as described, in at least one embodiment, heating element 410 is an elongate conductor (which may be hollow, solid, and/or in any other geometry). In some embodiments, heating element 106 may include a coiled wire, such as a coiled copper wire, and/or any other thermally and electrically conductive material, such as steel, iron, and the like. More generally, heating element 106 may be selected based upon thermally as well as electrically conductive properties, where it is desirable, in at least some embodiments, that heating element be both thermally and electrically conductive. In some embodiments, a non-corrosive and/or biologically inert metal may be selected for heating element 106, such as for example, if heating element 106 contacts liquid within fluid warming container 100. In some embodiments, heating element 106 may be coated or sheathed in a heat resistant coating, such as a heat-resistant shrink-wrap coating, and the like.

FIG. 2 is a cross-sectional view of container 100 taken along section 2-2 of FIG. 1. In the cross-sectional view, insulating portion 112, base portion 102, sidewall portion 104, and heating element 106 can be seen. In addition, as shown, in at least some embodiments, handle 110 may define a receptacle 136. In at least some embodiments, a power source 138 may be positioned and/or secured within receptacle 136. In at least one embodiment, power source 138 is any energy storage device, such as a battery. In some embodiments power source 138 may include a primary or non-rechargeable battery, such as an alkaline battery and/or any other suitable non-rechargeable battery cell type. In some embodiments, power source 138 may include a rechargeable battery, such as a lithium-ion battery and/or any other suitable rechargeable battery cell type. Where power source 138 is rechargeable, an electrical charging port may be included, such as a USB-C charging port and/or any other suitable charging port. In some embodiments, power source 138 may include a capacitor, and/or any other energy storage element that may be discharged to provide a flow of electrical current. Accordingly, as described herein, fluid warming container 100 is self-powered and can be used to warm liquid without plugging container 100 into a wall outlet and/or otherwise connecting container 100 to an external power source.

It may, in at least some embodiments, be desirable to house power source 138 within handle 110 to maintain a separation between heating element 106 and power source 138, such as for safety reasons. However, in some embodiments, power source 138 may not be in handle 110 but in another region or receptacle of container 100, such as within a receptacle defined by base portion 102 (e.g., a receptacle within, under, and/or otherwise proximate base portion 102), and the like.

As described in additional detail herein, power source 138 may be electrically connected through switch 108 to heating element 106, where switch 108 may be selectively actuated using pushbutton actuator 134 to facilitate the flow of electrical current between power source 138 and heating element 106 to energize and de-energize heating element 106.

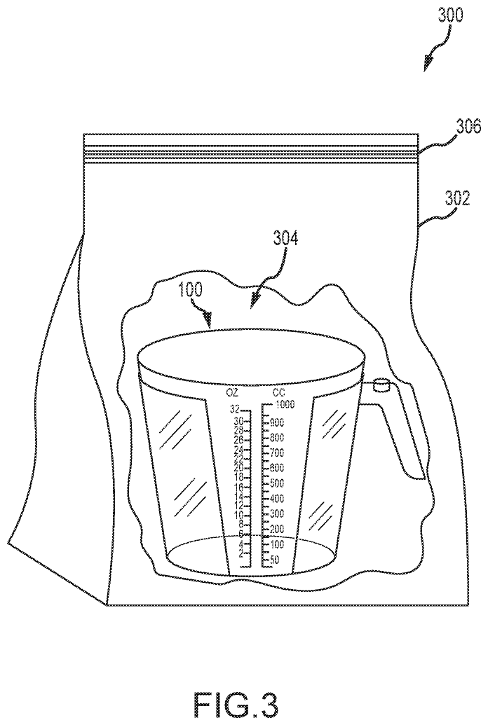

FIG. 3 is a perspective view of an example embodiment of a package 300 containing fluid warming container 100 (shown in FIG. 1), in which a portion of package 300 is cutaway to show fluid warming container 100 inside. Accordingly, in the example embodiment, package 300 includes a casing 302 that defines a sterile, sanitized, or otherwise disinfected interior region 304.

In various embodiments, interior region 304 may be sterilized or disinfected prior to receiving fluid warming container 100 and sealed, such as at a first end 306 to enclose and seal fluid warming container 100 therein. In addition, as described herein, fluid warming container 100 may in some embodiments be sterilized or disinfected using any suitable known technique prior to placement within package 300. In some embodiments, interior region 304 may be filled with a sterilizing or disinfecting gas or substance, such as ozone, plasma gas, vaporized hydrogen peroxide, ethylene oxide (EO or EtO), and/or any other suitable sterilizing composition. Fluid warming container 100 may thus undergo sterilization or disinfecting within package 300. In some embodiments, container 100 may be disinfected or sterilized prior to placement within package 300 as well as once package 300 has been sealed, such as using any of the disinfecting compositions described herein. In addition to these disinfecting compositions, other disinfecting processes and compositions may be used such as, for example, but not limited to, sterilization using ultraviolet light, heat sterilization, and the like.

As described elsewhere herein, fluid warming container 100 may be shipped and stored indefinitely inside package 300. To facilitate a surgical procedure, fluid warming container 100 may be unsealed from package 300 only within a sterile surgical environment (e.g., on a sterile surgical field), such that a medical care professional is assured that fluid warming container is properly sterilized prior to use and has not been stored or unsealed in any non-sterile environment.

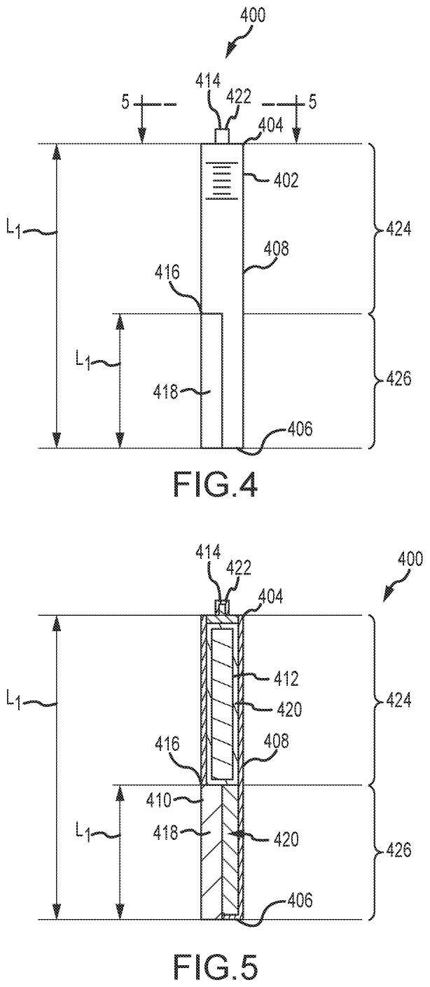

FIG. 4 is a perspective view of an example embodiment of a fluid warming device 400. FIG. 5 is a cross-sectional view of fluid warming device 400 taken along section 5-5 of FIG. 4.

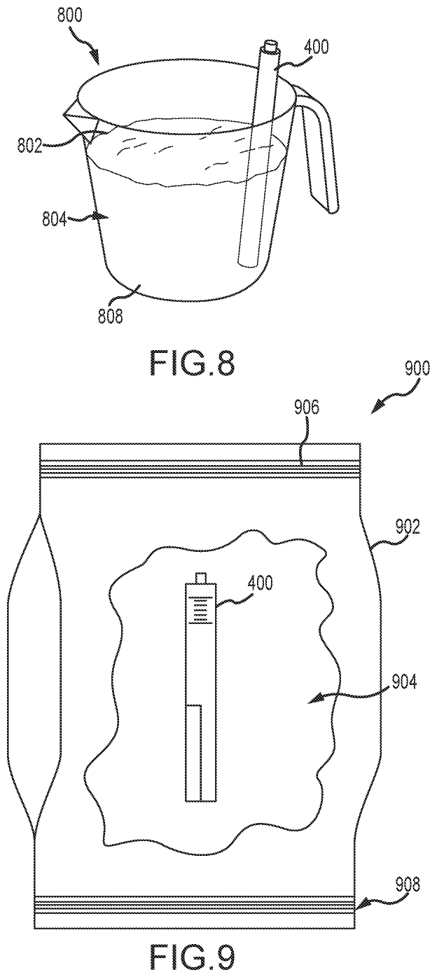

Fluid warming device 400 may, like fluid warming container 100, be used to heat liquid to a desired temperature. For example, in at least some embodiments (see also FIG. 8), fluid warming device 400 may be placed (e.g., vertically and/or at an angle) within a container 800, such as any suitable medical container (e.g., a graduated medical container). Fluid warming device 400 may be activated, as described in additional detail herein, to heat the liquid within the selected container 800 for use during a surgical procedure. As a result, in at least some embodiments, fluid warming device 400 may be used as an alternative to fluid warming container 100. Specifically, fluid warming device 400 may be used with any suitable container 800 (e.g., any standard container that excludes self-heating functionality) to heat liquid received within container 800.

In some embodiments, fluid warming device 400 can also be used with fluid warming container 100, such as for example, during a protracted or very long surgery, to maintain a temperature of the liquid, and the like. However, in general terms, the embodiments described herein offer a treating physician or surgeon several options, including fluid warming container 100 as well as fluid warming device 400, both of which may function independently of one another to heat a volume of liquid to a desired temperature, such as for example, a temperature in a selected or predefined range of human body temperature (e.g., in any desired range of 98.6 degrees Fahrenheit, such as for example, but not limited to, plus or minus 5 degrees Fahrenheit, plus or mins 10 degrees Fahrenheit, etc.)

Accordingly, in the example embodiment, fluid warming device 400 can include a housing 402 having a first end 404, a second end 406, and a body 408 extending therebetween. In addition, and as described in additional detail herein, fluid warming device 400 includes, in the example embodiment, a heating element 410, a power source 412, and a switch 414. In various embodiments, power source 412 may be substantially the same as power source 138 and may include any suitable energy storage device, such as any rechargeable or non-rechargeable battery.

In various embodiments, one or more portions of fluid warming device 100, such as housing 402, may be composed of any suitable plastic or synthetic material, such as, without limitation, polyethylene terephthalate (PET), high density polyethylene, (HDPE), low density polyethylene (LDPE), polypropylene (PP), and/or any other biologically safe or inert plastic or composition of plastics, such as any plastic suitable for contact with a human body and/or liquids that may be heated by fluid warming device 400. In addition, the plastics used may be transparent, translucent, opaque, and/or not transparent or translucent.

In some embodiments, the plastic or plastic composition used in the manufacture of fluid warming device 400 may be heat resistant to a predefined temperature, such that fluid warming device 400 (e.g., housing 402) does not melt when heating element 410 is active, as described further herein. Stated another way, in some embodiments, at least a portion of fluid warming device 400 may be composed of a heat resistant plastic and/or another material, such as stainless steel or another metal. However, in other embodiments, heating element 410 may not reach temperatures capable of melting housing 402, and housing may be made from any biologically suitable plastic.

In the example embodiment, heating element 410 is disposed at least partially within housing 402. For example, in at least one embodiment, housing 402 includes an aperture 416 in body 408 thereof. At least a portion 418 of heating element 410 may be positioned within aperture 416, such that portion 418 of heating element 410 is exposed to fluid contact when fluid warming device 400 is arranged within a medical container containing fluid.

To facilitate this feature, aperture 416 may occupy a lower region of housing 402, such as a lower one-half of housing 402, a lower one-quarter of housing 402, and the like. Stated another way, in at least one embodiment, aperture 416 may be positioned in a lower region of housing 402 to facilitate contact with liquid when device 400 is placed within container 800, where second end 406 may generally rest or contact a bottom surface of container 800 to support device 400 at rest. In some embodiments, aperture 416 may occupy any other region or area of housing 402, such as any region between first end 404 and second end 406, up to and including all or a portion of an entire length, L1, of housing 402 between first end 404 and second end 406.

In addition, in various embodiments, first end 404 and second end 406 may be substantially circular to define a substantially cylindrical body 408 therebetween. However, other shapes are contemplated by and within the scope of the present disclosure.

In at least some embodiments, aperture 416 may extend over any portion of the circumference and/or area of housing 402. For example, if first end 404 and second end 406 are generally circular to define a substantially cylindrical housing 402, aperture 416 may extend to include any measure of the full three-hundred-and-sixty degrees comprising the circumference of housing 402. Likewise, as described herein, aperture 416 may extend any distance between first end 404 and second end 406 to define an aperture length L2, to thereby define an aperture 416 of any desired dimensions, both in terms of length, L2, as well as circumference.

In the example embodiment, housing 402 may define an interior 420 that may house a variety of components, such as in a water-tight or sealed configuration. For example, in at least one embodiment, heating element 410 and power source 412 may be disposed within interior 420. Likewise, at least a portion of switch 414 may extend into interior 420, while another portion of switch 414 extends outside interior 420 and is accessible by a user (e.g., for pressing a pushbutton actuator 422 of switch 414).

In addition to these features, in at least some embodiments, power source 412 may be disposed in a first region 424 of housing 402, and heating element 410 may be disposed in a second region 426 of housing 402. First region 424 may be separate from and/or nonoverlapping with second region 426 to ensure that power source 412 is thermally isolated from heating element 410. For example, as shown in the example of FIG. 5, power source 412 may occupy an upper area (corresponding to first region 424) within interior 420. Likewise, heating element may occupy a lower area (corresponding to second region 426) within interior 420.

During operation, as described herein, device 400 may be inserted in a liquid second end 406 down (e.g., in contact with a bottom surface of medical container 800), such that heating element 410 is submerged, at least partially, in the liquid. As a result, the heat or thermal energy generated by heating element 410 may be more efficiently radiated away from power source 412 and into the surrounding liquid. In some embodiments, an insulation layer may be positioned between power source 412 and heating element 410 to further reduce thermal bleed toward power source 412 during operation.

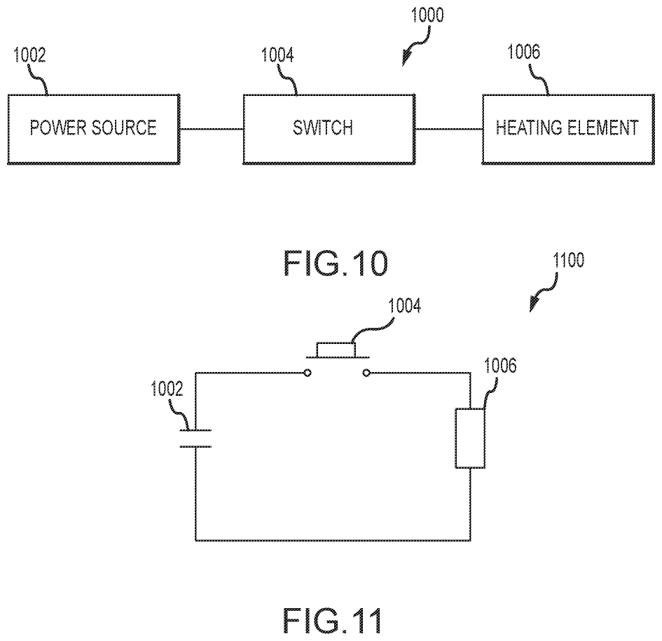

As described in additional detail herein (e.g., with reference to FIGS. 10-12), in the example embodiment, power source 412 is electrically connected to heating element 410 via switch 414, where switch 414 may be selectively actuated to facilitate the flow of electrical current between power source 412 and heating element 410 to energize and de-energize heating element 410.

In addition, in one or more embodiments, heating element 410 may include a substantially elongate metallic conductive member. When electrical current flows through heating element 410, a temperature of heating element 410 may increase to raise the temperature of a liquid in contact with device 400.

For example, in at least some embodiments heating element 410 may include any of a variety of metal conductors in any of a variety of suitable geometries. For example, as described, in at least one embodiment, heating element 410 is an elongate conductor (which may be hollow, solid, and/or in any other geometry). In some embodiments, heating element 410 may include a coiled wire, such as a coiled copper wire, and/or any other thermally and electrically conductive material, such as steel, iron, and the like. More generally, heating element 410 may be selected based upon thermally and/or electrically conductive properties, where it is desirable, in at least some embodiments, that heating element be both thermally and electrically conductive. In some embodiments, a non-corrosive and/or biologically inert metal may be selected for heating element 410.

In some embodiments, a diameter of heating element 410 is substantially the same as a diameter defined by second end 406 of housing 402. In some embodiments, a diameter of heating element 410 is less than the diameter defined by second end 406. Accordingly, heating element 410 may include a variety of dimensions. In at least one embodiment, heating element 410 occupies substantially all of second region 426. In another embodiment, heating element 410 occupies less than the entirety of second region 426, such as one-half, one-quarter, and the like. In the latter case, any leftover or unused spaced within region 426 may be filled with insulation, electrical wires or other electrical components, and the like.

In at least one embodiment, housing 402 excludes aperture 416. For example, housing 402 may, in at least one embodiment, fully contain heating element 410. In such an embodiment, heating element 410 may conduct thermal energy through housing 402, which may itself be thermally conductive and/or heat resistant, as described. For example, in place of aperture 416, housing 402 may contain a metallic region that corresponds to and is in contact with heating element 410. In another example, housing 402 may include a thermally conductive and/or heat-resistant plastic in contact with heating element 410.

In some embodiments, as described in additional detail herein, heating element 410 may be configured to peak at a preselected maximum temperature, which may be set to prevent overheating housing 402 as well as to prevent overheating liquid in contact with fluid heating device 400. It may for example be useful to prevent heating the liquid beyond a certain threshold temperature (e.g., 101 degrees Fahrenheit) to ensure that device 400 cannot heat liquid to an undesired temperature. To achieve the latter result, as described herein, fluid warming device 400 (as well as fluid warming device 600 and/or fluid container 100 in other embodiments), may include a temperature sensor, a power disconnect switch, an audible or visible alarm, and/or other circuitry configured to interrupt or facilitate interruption of electrical power when the liquid reaches a threshold temperature.

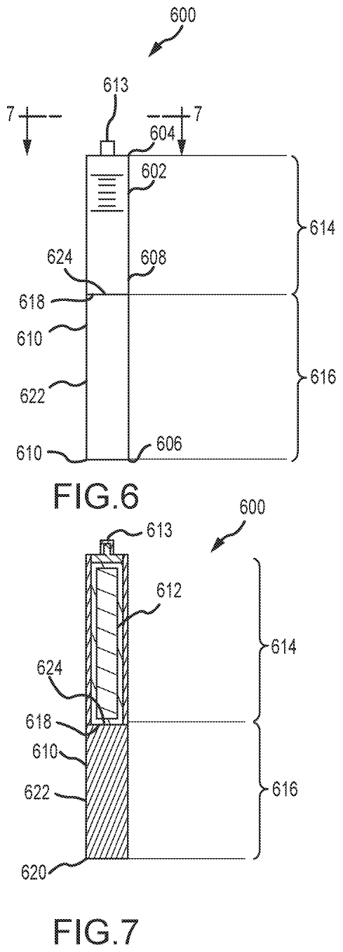

FIG. 6 is a perspective view of an alternate example embodiment of a fluid warming device 600. FIG. 7 is a cross-sectional view of fluid warming device 600 taken along section 7-7 of FIG. 6. In various embodiments, fluid warming device 600 may include a housing 602 having a first end 604, a second end 606, and a body 608 extending therebetween. In addition, and as described in additional detail herein, fluid warming device 600 may include a heating element 610, a power source 612, and a switch 613.

Accordingly, in the example embodiment, fluid warming device 600 is substantially the same as fluid warming device 400, except, as described, that heating element 610 occupies and defines an entire second region 616. Specifically, in this embodiment, heating element 610 includes a first end 618, a second end 620, and an elongate body 622 extending therebetween. In at least one embodiment, heating element 610 is a solid elongate member, such as a solid elongate metallic member. In at least one embodiment, heating element 610 is hollow and defines an interior region, which may be filled with insulation, electrical components, such as electrical connections between a power source 612 and heating element 610, and the like. In these embodiments, first end 618 of heating element 610 may be coupled to an engagement surface or engagement end 624 of housing 602, such that housing 602 occupies first region 614 and heating element 610 occupies second region 616.

In addition, as described herein, heating element 610 may include any of a variety of metal conductors in any of a variety of suitable geometries. For example, as described, in at least one embodiment, heating element 610 is an elongate conductor (which may be hollow, solid, and/or in any other geometry). In some embodiments, heating element 610 may include a coiled wire, such as a coiled copper wire, and/or any other thermally and electrically conductive material, such as steel, iron, and the like. More generally, heating element 610 may be selected based upon thermally and/or electrically conductive properties, where it is desirable, in at least some embodiments, that heating element be both thermally and electrically conductive.

FIG. 8 is a perspective view of an example embodiment of a medical container 800 that includes fluid warming device 400 (shown in FIGS. 4-7). It will be appreciated that fluid warming device 600 may also be used with medical container 800. In the example embodiment, medical container 800 is any container that may be used to contain and pour a liquid, as described herein, during a medical procedure, such as a surgery. However, it will be appreciated that fluid warming device 400 may be used more broadly in any application where it is desirable to heat or warm a volume of liquid, such as in kitchen and/or in any other setting.

Accordingly, as shown, fluid warming device 400 can be placed within container 800 in a substantially vertical orientation, such that first end 404 of housing 402 extends at least above a level 802 of liquid 804 within container 800, and such that second end 406 of housing 402 is submerged within liquid 804. In addition, as shown, second end 406 may rest on a base portion 808 of container 800. In some embodiments, a clip or other fastener may be provided to removable or detachably couple fluid warming device 400 to and edge or sidewall portion of container 800. In some embodiments, a nonslip portion, such as a rubber pad, may be coupled to second end 406 of housing 402 to increase a friction between base portion 808 and fluid warming device 400 and to minimize, reduce, or eliminate motion or sliding of device 400 when device 400 is placed within container 800.

FIG. 9 is a perspective view of an example embodiment of a package 900 containing fluid warming device 400 (shown in FIGS. 4 and 5), in which a portion of package 900 is cutaway to show fluid warming device 400 inside package 900. It will be appreciated that fluid warming device 600 can also be contained inside package 900. Accordingly, in the example embodiment, package 900 includes a casing 902 that defines a sterile, sanitized, or otherwise disinfected interior region 904.

In various embodiments, interior region 904 may be sterilized or disinfected prior to receiving fluid warming device 400 and sealed, such as at a first end 906 and/or at a second end 908 to enclose and seal fluid warming device 400 therein. In addition, as described herein, fluid warming device 400 may in some embodiments be sterilized or disinfected using any suitable known technique prior to placement within package 900. In some embodiments, interior region 904 may be filled with a sterilizing or disinfecting gas or substance, such as ozone, plasma gas, vaporized hydrogen peroxide, ethylene oxide (EO or EtO), and/or any other suitable sterilizing composition. Fluid warming device 400 may thus undergo sterilization or disinfecting within package 900. In some embodiments, fluid warming device 400 may be disinfected or sterilized prior to placement within package 900 and/or once package 900 has been sealed, such as using any of the disinfecting compositions described herein. In addition to these disinfecting compositions, other disinfecting processes may be used in addition, or in the alternative, such as, for example, but not limited to, sterilization using ultraviolet light, heat sterilization, and the like.

As described elsewhere herein, fluid warming device 400 may be shipped and stored indefinitely inside package 900. To facilitate a surgical procedure, fluid warming device 400 may be unsealed from package 900 only within a sterile surgical environment (e.g., on a sterile surgical field), such that a medical care professional is assured that fluid warming container is properly sterilized prior to use and has not been stored or unsealed in any non-sterile environment. In some embodiments, fluid warming device 400 may also be packaged together with medical container 800 and/or in some embodiments, with fluid warming container 100. As a result, in some embodiments, package 900 may contain any of fluid warming device 400, medical container 800, and/or fluid warming container 100.

FIG. 10 is a block diagram of an example embodiment of a system 1000 for use with fluid warming container 100 (shown in FIGS. 1 and 2) and/or fluid warming devices 400 and/or 600 (shown in FIG. 4-7). FIG. 11 is a diagram of an electrical circuit 1100 of system 1000 shown in FIG. 10.

Accordingly, in the example embodiment, system 1000 includes a power source 1002, a switch 1004, and/or a heating element 1006. In various embodiments, power source 1002 may correspond to any of the power sources described herein, such as any of power sources 138, 412, and/or 616. Likewise, switch 1004 may correspond to any of the switches described herein, such as any of switches 108, 414, and/or 613. Heating element 1006 may correspond to any of heating elements 106, 410, and/or 610.

In operation, a user may activate switch 1004, as shown, to complete an electrical circuit between power source 1002 and heating element 1006, which includes an electrical resistance and which it will be appreciated may release thermal energy as electrical current flows through the electrical load comprising heating element 1006. In various embodiments, switch 1004 includes a pushbutton switch (e.g., a pushbutton switch having a pushbutton actuator, such as pushbutton actuator 134).

In some embodiments, power source 1002 includes a battery, such as any suitable rechargeable and/or non-rechargeable electrochemical cell, as described herein. In at least one embodiment, power source 1002 includes a battery that is pre-charged as desired. In some embodiments, power source 1002 includes a battery that is pre-charged and calibrated in accordance with a resistance of heating element 1006 to include an electrical charge sufficient, at least, to warm a pre-defined volume of liquid (e.g., 1 liter) to a pre-defined temperature, or to within a pre-defined temperature range (e.g., 98.6 degrees Fahrenheit plus or minus 1, 2, 3, 4, or 5 degrees Fahrenheit and/or any other desired threshold value). In some embodiments, power source 1002 includes an electrical charge sufficient, at least, to maintain a pre-defined volume of liquid at a pre-defined temperature, or within a pre-defined range of temperatures, for a pre-defined period of time, such as 1 hour, 2 hours, 3 hours, 4 hours, 5 hours, and/or any other suitable period of time, such as any period of time that may correspond to a period of time associated with a given surgical procedure. As a result, container 100 as well as devices 400 and 600 may facilitate warming a volume of liquid and keeping the liquid warm during the entirety of the procedure, such that it is not necessary to discard the liquid, since it is not permitted to cool until the procedure is completed.

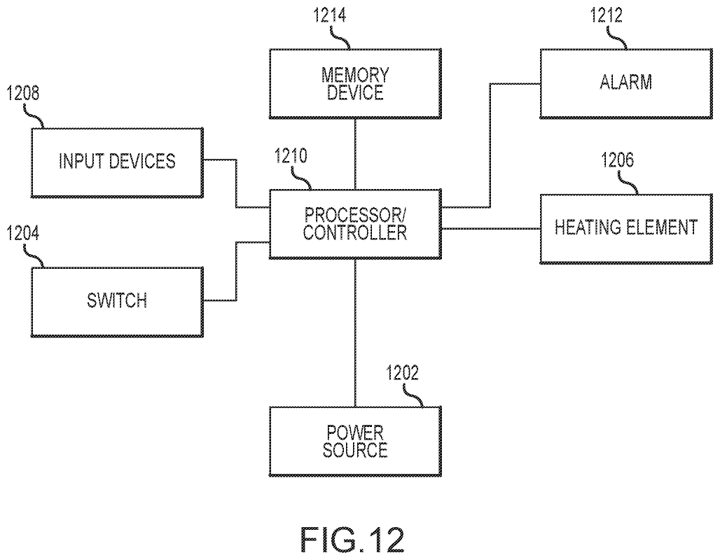

FIG. 12 is a block diagram of an alternate example embodiment of a system 1200 for use with fluid warming container 100 (shown in FIGS. 1 and 2) and/or fluid warming devices 400 and/or 600 (shown in FIG. 4-7). In the example embodiment, system 1200 may include a power source 1202, a switch 1204, a heating element 1206, one or more input devices 1208, a processor or controller 1210, an alarm 1212, and/or a memory device 1214, such as any suitable tangible, non-transitory, computer-readable storage medium having instructions stored thereon that are executable by controller 1210 to perform any of the functions described herein.

In various embodiments, power source 1202 may correspond to any of the power sources described herein, such as any of power sources 138, 412, and/or 616. Likewise, switch 1204 may correspond to any of the switches described herein, such as any of switches 108, 414, and/or 613. Heating element 1206 may correspond to any of heating elements 106, 410, and/or 610.

Input devices 1208 may include any of a variety of input devices and/or peripheral devices, such as for example, any suitable temperature sensor, any thermistor (e.g., a dial) and/or other device for adjusting a resistance and/or temperature of heating element 1206, any timer for setting a warming period and/or an “on” or “active” period, during which heating element 1206 is set to generate heat, and the like.

Controller 1210 may include any suitable computer processor and/or controller, such as any microcontroller, a microcomputer, any programmable logic controller (PLC), any application specific integrated circuit, any other programmable circuit, and/or in general, any computer processor or processing device, such as, but not limited to, those which may be commercially available “off the shelf.”

Alarm 1212 may include any suitable audible and/or visible alarm, such as without limitation, any audio speaker or illumination element (e.g., a light emitting diode or LED). Alarm 121 may be configured to generate a sound or audible alarm and/or a visible alarm in response to, for example, controller 1210 determining that a pre-defined or user selected temperature has been achieved based on temperature measurement data obtained from an input device 1208 that includes a temperature sensor. In some embodiments, alarm 1212 may also be configured to generate a warning alarm in response to controller 1210.

Accordingly, and in more general terms, any of the fluid warming container 100 and/or fluid warming devices 400 and/or 600 may be configured and/or programmed to sense a temperature of liquid, such as liquid contained within container 100 and/or container 800. In some embodiments, when the temperature of the liquid reaches a pre-defined temperature, which may be user selected and/or pre-set (e.g., 98.6 degrees Fahrenheit), controller 1210 may control alarm 1212 to signal that the desired temperature has been achieved. In response, a user, such as a surgeon or nurse, may use the warmed liquid during a surgery to rinse a human body cavity and/or as otherwise desired.

Controller 1210 may also control a flow of electrical current to heating element 1206 to maintain a temperature of the liquid at or within a desired range of the pre-defined or pre-set temperature, which may facilitate keeping the liquid at the desired temperature for the duration of a surgical procedure without having to be concerned that the liquid has cooled or overheated. In some embodiments, an input device 1208 comprising a temperature sensor may include an analog and/or digital thermometer and/or another temperature readout device (e.g., a display device), which may output a temperature measurement for viewing by a surgeon or other medical staff, and which may be used to visually verify that a temperature of liquid heated by devices 100, 400, and/or 600 is acceptable (e.g., within a desired range of human body temperature) prior to using the liquid during a surgical procedure. In at least one embodiment, system 1200 may include a wireless interface, such as a BLUETOOTH and/or WIFI transmitter and/or transceiver, that facilitates transmitting temperature and/or other data from container 100 and/or devices 400 and 600 to a computer-assisted surgical system in the operating room.

In addition, in some embodiments, memory device 1214 may store one or more heating modes, each of which may be associated with a pre-defined temperature and/or a pre-defined temperature range. For example, a first mode may be associated with temperatures in the range of 90-93 degrees Fahrenheit, a second mode may be associated with temperatures in the range of 94-97 degrees Fahrenheit, a third mode may be associated with temperatures in the range of 98-101 degrees Fahrenheit, and the like. It will be appreciated that these ranges are merely examples and that other ranges are contemplated by and within the scope of the present disclosure. During operation, a user may select a desired mode, such as using switch 1204. For example, a first selection of switch 1204 (e.g., by depressing the pushbutton actuator) may result in a selection of the first mode, a second selection of switch 1204 (e.g., depressing switch 1204 twice) may result in a selection of the second mode, and so on. Thus, a user may cycle through modes by repeatedly depressing the actuator. In some embodiments, memory device 1214 may also store time periods, which may be user selectable, such as using switch 1204 as described and/or using any other timer selection input device. In one example, if a surgical procedure is expected to continue for two hours, a user may select a period of two hours, or two- and one-half hours, and controller 1210 may control heating element 1206 to warm liquid to a pre-defined and/or pre-set temperature for the selected period.

Accordingly, in operation, and in various embodiments, fluid warming container 100 and/or fluid warming devices 400 and/or 600 may be used to warm a volume of liquid, such as water, saline, medicated fluids, and/or any other fluid that may be used during a surgical procedure, to a desired temperature. Specifically, as described herein, it is often desirable during a surgical procedure to irrigate and/or lavage a surgical wound with water or saline solution. Because many surgical procedures maintain a patient's body temperature at or near normal human body temperature (e.g., 98.6 degrees Fahrenheit), it is also desirable to pre-warm the liquid to the same temperature, or within a desired or acceptable range thereof. As a result, liquid may be poured or scooped into container 100 and/or a standard medical container 800 and heated or warmed to a desired temperature by container 100 itself and/or in some embodiments, by fluid warming devices 400 and/or 600.

It will also be appreciated that other mammals, such as cats, dogs, as well as a variety of larger breed animals, frequently undergo surgery, and that these creatures may have nominal body temperatures that are different from human body temperature. As a result, container 100 and/or devices 400 and 600 may also be programmed or pre-set to bring a liquid to a temperature closer to the body temperature of the animal prior to performing surgery on the animal. Stated another way, container 100 and devices 400 and 600 are useful for performing surgery on any mammal and can be adjusted accordingly.

Container 100 and devices 400 and 600 may also be adjusted and used in other applications, such as in a kitchen to bring liquid to a desired temperature, and the like. In some embodiments, container 100 and/or devices 400 and/or 600 may be configured to cool a liquid, such as using one or more thermoelectric cooling devices (e.g., Peltier coolers and/or thermoelectric heat pumps) in place of and/or in addition to any of the heating elements 1206 described herein.

In addition to these features, container 100 and/or devices 400 and 600 may be sterilized or disinfected and packaged in sterile or disinfected packaging, as described herein, during a manufacturing or packaging process. Prior to use during a surgical procedure, a surgeon, a nurse, or other medical personnel may unseal container 100 and/or devices 400 and 600, as desired, in a sterile surgical setting, such as by unsealing the container 100 and/or devices 400 and/or 600 on a sterile surgical field. This process may ensure that container 100 and devices 400 and 600 have not encountered any non-sterile environment prior to use.

Finally, fluid warming container 100 and/or a standard medical container 800 may receive a desired liquid, as described herein. In some embodiments, the liquid is poured from a room temperature or heated bottle into containers 100 and/or 800. In other embodiments, containers 100 and/or 800 may be used to scoop the liquid from a tub or basin situated within an operating room. In the case of fluid warming container 100, a user may activate container 100 by pressing pushbutton actuator 134 of switch 108, which may result in container 100 heating the liquid contained therein, as described herein. In the case of fluid warming devices 400 and/or 600, these may be placed by a user in container 100 and/or container 800 and activated (using respective switches 414 and/or 613) to heat or warm the liquid within container 100 and/or 800.

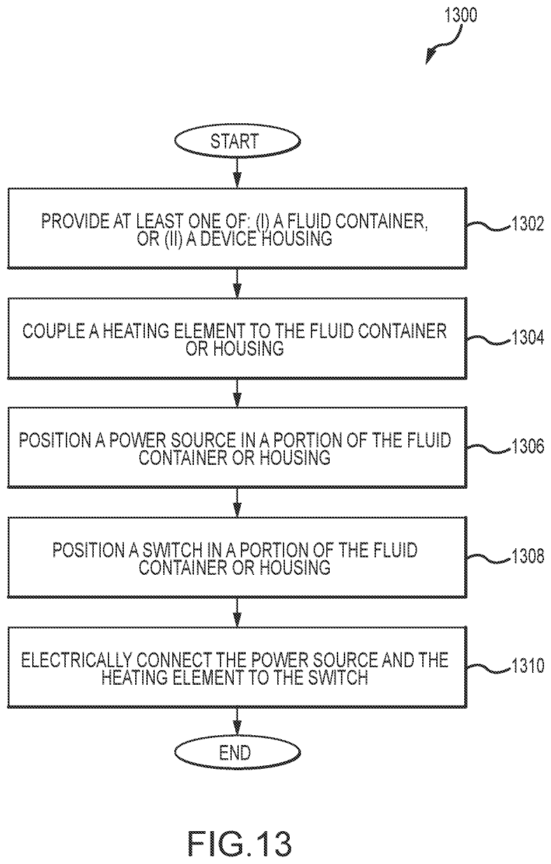

FIG. 13 is a flowchart illustrating an example process 1300 for manufacturing fluid warming container 100 (shown in FIGS. 1 and 2) and/or fluid warming devices 400 and/or 600 (shown in FIG. 4-7). In various embodiments, a fluid container and/or a device housing, such as housing 402, may be provided (step 1302). A heating element, such as heating element 106, may be coupled to container 100, or in the case of housing 402, a heating element such as heating element 410, may be coupled to housing 402 (step 1304). In at least one embodiment, a power source, such as power source 138, may be positioned and/or coupled in a portion of container 100, such as handle 110, and/or in the case of fluid warming device 400, power source 412 may be positioned and/or coupled within housing 402 (step 1306). A switch, such as switch 108 and/or switch 414, may be positioned in and/or coupled to a portion of container 100 and/or housing 402, respectively (step 1308). For example, switch 108 may be positioned such that pushbutton actuator 134 thereof is accessible outside handle 110 by a user. Likewise, a pushbutton actuator of switch 414 may extend from housing 402 in the same manner. Inside handle 110 and/or housing 402, it will be appreciated that a portion of the respective switch 108 and/or 414 may be stowed to form a portion of the electrical circuit (e.g., circuit 1100). In addition, in at least some embodiments, the respective power source 138 and/or 412, may be electrically connected, using a respective switch 108 and/or 414, to respective heating element 106 or 410 (step 1310).

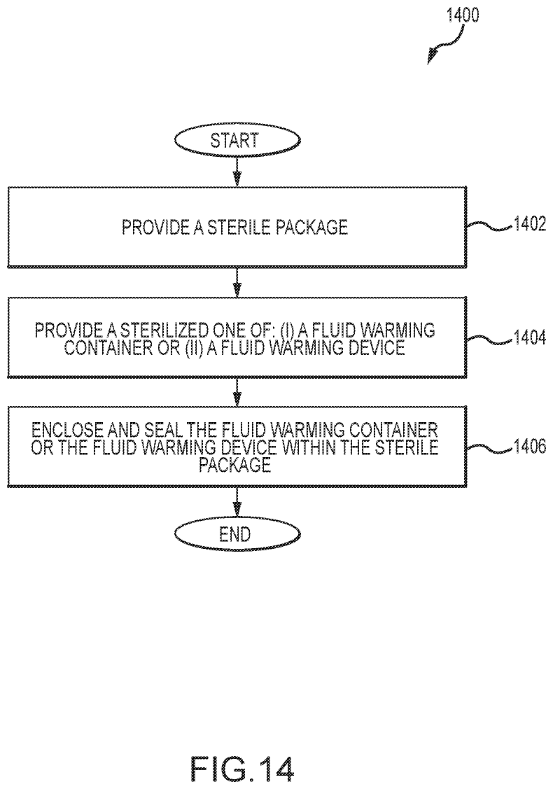

FIG. 14 is a flowchart illustrating an example process 1400 for packaging fluid warming container 100 (shown in FIGS. 1 and 2) and/or fluid warming devices 400 and/or 600 (shown in FIG. 4-7) in a sterile package. In the example embodiment, and as described herein, a package 300 and/or 900 is provided (step 1402). As described herein, container 100, devices 400 and/or 600, and/or an interior region 304 and/or 904 of packages 300 and/or 900 may be disinfected or sterilized, such as using a sterilizing or disinfecting gas or process, such as ozone, plasma gas, vaporized hydrogen peroxide, ethylene oxide (EO or EtO) processing, and/or any other suitable sterilizing composition or process (step 1404). In some embodiments, container 100 and/or devices 400 and/or 600 are sterilized within packages 300 and 900, respectively, using ethylene oxide injected or otherwise filled within packages 300 and 900. Fluid warming container 100 and/or devices 400 and/or 600 are also enclosed and sealed within the respective packages 300 and/or 900 (step 1406).

Embodiments of self-powered (e.g., battery-powered) fluid warming container and fluid warming device are thus described herein. In the example embodiments, the fluid warming container is arranged to receive and warm a fluid or liquid, such as liquid water, saline, combinations thereof, one or more medicaments and/or combinations of water, saline, and/or medicaments, and/or any other liquid that a user may desire to heat or warm to a particular temperature. Likewise, the fluid warming devices described herein may include a housing that defines elongate member, which is also configured to warm a volume of liquid, such as a volume of liquid contained within a standard medical container, to a desired temperature.

In various embodiments, it may, for example, be desirable to warm the liquid to within a predefined range of a mammalian body temperature prior to performing a surgical procedure on the mammal. In the case of a human patient, it may for instance be desirable to warm the liquid within the fluid warming container to about human body temperature (e.g., 98.6 degrees Fahrenheit). At least one advantage associated with warming the liquid to a temperature at or near human body temperature is, it will be appreciated, to bring the liquid to the same temperature as the patient's body prior to using the liquid during the surgery, where for example, it is common to use the liquid to rinse portions of the patient's anatomy that have been opened and/or exposed by the surgical procedure (e.g., during wound irrigation and/or lavage, as described herein).

Cold or room temperature liquids can be undesirable during surgical procedures for a variety of reasons, including that using liquids that are not sufficiently warm may cause stress on the patient's body (which is already stressed by the surgery itself). Another technical advantage of the present disclosure is thus that the fluid warming container and/or fluid warming devices described herein are useful to alleviate surgical stress on a patient as well as to promote healing. Likewise, embodiments of the present disclosure facilitate the introduction of sterilized and safe liquids during a surgical procedure, which may also be reliably controlled to achieve and maintain a desired temperature close to a mammalian body temperature.

To facilitate a sterile surgical field, the fluid warming container and/or fluid warming devices described herein may be sterilized or disinfected and/or packaged in sterilized or disinfecting packages. For example, in some embodiments, ethylene oxide may be introduced in a package together with a container and/or device, and the package subsequently sealed, to facilitate and promote sterilization and/or disinfection of the container and/or device inside the package. The container and/or device may be stored inside of the package for any duration of time in the sterile and/or disinfected state until it is desired to unseal the container and/or devices for use during a surgery.

For example, in at least one embodiment, a member of a surgical team may unseal a sterilized and/or disinfected container and/or device by opening the sealed packaging and placing the container or device on a sterile surgical field, such as on a sterile tray or work surface within a draped out and sterilized operating room. Another technical improvement and advantage of the embodiments described herein is thus that medical staff can ensure that the container or device is sterile when it is finally unsealed and placed within the sterile surgical field, since it has remained sealed until that time.

In addition to these features and improvements, the containers and devices described herein may include a standalone energy storage device, such as a rechargeable and/or non-rechargeable battery. The battery may be included in the container and devices during manufacturing, such that the container and devices are self-powered and operable “out of the box” without any necessity of charging the battery prior to use.

Moreover, the batteries used in the various embodiments may be pre-charged to include an electrical charge sufficient at least to warm a specified volume of liquid to a desired temperature (e.g., at or near a mammalian body temperature) and/or, in addition, to maintain the liquid at the desired temperature for the duration of a surgical procedure. The containers and devices described herein may also be used repeatedly during a surgical procedure to warm consecutive volumes of liquid, such as for example, after a preceding volume of liquid is warmed and used as part of the procedure.

In some embodiments, and in addition to the many other advantages described herein, the containers and devices disclosed may be set, such as by a user, to bring liquid to a desired temperature for any desired period of time as well as, in some cases, to prevent overheating the liquid beyond an established maximum temperature. As a result, safety, efficiency, and overall performance are further improved by these and other features.

Moreover, the batteries used in the various embodiments may be pre-charged to include an electrical charge sufficient at least to warm a specified volume of liquid to a desired temperature (e.g., at or near a mammalian body temperature) and/or, in addition, to maintain the liquid at the desired temperature for the duration of a surgical procedure. The containers and devices described herein may also be used repeatedly during a surgical procedure to warm consecutive volumes of liquid, such as for example, after a preceding volume of liquid is warmed and used as part of the procedure. In some embodiments, and in addition to the many other advantages described herein the containers and devices disclosed may be set, such as by a user, to bring liquid to a desired temperature for any desired period of time as well as, in some cases, to prevent overheating the liquid beyond an established maximum temperature. As a result, safety, efficiency, and overall performance are further improved by these and other features.

As used herein, the terms “controller,” “processor,” “computer,” and related terms, e.g., “processing device” and “computing device,” are not limited to just those integrated circuits referred to in the art as a computer, but broadly refers to a microcontroller, a microcomputer, a programmable logic controller (PLC), an application specific integrated circuit, and other programmable circuits, and these terms are used interchangeably herein. In the embodiments described herein, memory includes, but is not limited to, any non-transitory computer-readable storage medium, such as, but not limited to, random-access memory (RAM), and any computer-readable non-volatile storage medium, such as flash memory.

Example embodiments of devices to and related components are described above in detail. The devices are not limited to the specific embodiments described herein, but rather, components of systems and/or steps of the methods may be utilized independently and separately from other components and/or steps described herein. For example, the configuration of components described herein may also be used in combination with other processes and is not limited to practice with the systems and related methods as described herein. Rather, the example embodiments can be implemented and utilized in connection with many applications where fluid warming are generally desired.

Although specific features of various embodiments of the present disclosure may be shown in some drawings and not in others, this is for convenience only. In accordance with the principles of the present disclosure, any feature of a drawing may be referenced and/or claimed in combination with any feature of any other drawing.

This written description uses examples to disclose the embodiments of the present disclosure, including the best mode, and also to enable any person skilled in the art to practice the disclosure, including making and using any devices or systems and performing any incorporated methods. The patentable scope of the embodiments described herein is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A fluid warming container comprising:
a base portion;
a sidewall portion coupled at a first end thereof to the base portion and extending away from the base portion to define an interior volume of the fluid warming container, the interior volume arranged to receive a liquid;
a heating element coupled to one of i) the base portion or ii) the sidewall portion;
a power source; and
a switch electrically connected to the power source and the heating element, the switch configured to selectively control a flow of electrical current from the power source to the heating element, whereby when the heating element receives the flow of electrical current, a temperature of the heating element increases.

2. The fluid warming container of claim 1, wherein the sidewall portion includes an inner surface and an outer surface, the fluid warming container further comprising an insulation layer coupled at least partially about the outer surface of the sidewall portion.

3. The fluid warming container of claim 2, wherein the outer surface of the sidewall portion includes a plurality of measuring indicia, and wherein the insulation layer is excluded from covering measuring indicia.

4. The fluid warming container of claim 1, wherein the sidewall portion is substantially transparent and includes a plurality of measuring indicia, the fluid warming container further comprising an insulating portion coupled to a portion of the sidewall to define a gap through which the plurality of measuring indicia are visible.

5. The fluid warming container of claim 1, further comprising a handle coupled to and extending from the sidewall portion, the handle defining a receptacle that houses the power source.

6. The fluid warming container of claim 5, wherein the power source is a battery, and wherein the switch includes a pushbutton actuator included in the handle and configured to be actuated by a user holding the fluid warming container by the handle.

7. The fluid warming container of claim 6, wherein the battery is pre-charged prior to introducing the fluid warming container within a sterile package.

8. The fluid warming container of claim 7, wherein the battery is pre-charged and calibrated in accordance with a resistance of the heating element to include at least a charge sufficient to warm a pre-defined volume of the fluid to a pre-defined temperature.

9. The fluid warming container of claim 8, wherein the pre-defined temperature is within 5 degrees Fahrenheit of 98.6 degrees Fahrenheit.

10. The fluid warming container of claim 1, further comprising a package including a casing, the casing defining a sterile interior region, the package enclosing and sealing the fluid warming container within the sterile interior region prior to use.

11. A fluid warming device comprising:
an elongate housing having a first end, a second end, and a body extending therebetween;
a heating element coupled to the housing;
a power source disposed within the housing; and
a switch electrically connected to the power source and the heating element, the switch configured to selectively control a flow of electrical current from the power source to the heating element, whereby when the heating element receives the flow of electrical current, a temperature of the heating element increases.

12. The fluid warming device of claim 11, wherein the switch includes a pushbutton actuator configured to be actuated by a user holding the fluid warming device.

13. The fluid warming device of claim 11, wherein the body of the housing defines an aperture, and wherein at least a portion of the heating element is included within the aperture, such that the portion of the heating element is exposed to contact with a liquid when the fluid warming device is positioned in a medical container containing the liquid.

14. The fluid warming device of claim 11, wherein the heating element includes a first end, a second end, and an elongate body extending therebetween, and wherein the first end of the heating element is coupled to the second end of the housing, such that heating element substantially depends from the housing.

15. The fluid warming device of claim 11, further comprising a processor and a tangible, non-transitory, computer-readable memory device having instructions stored thereon which when executed by the processor to receive a user selection of a warming mode and in response to receiving the selection of the warming mode to control a temperature of the heating element.

16. The fluid warming device of claim 11, wherein the power source is a battery.

17. The fluid warming device of claim 16, wherein battery is pre-charged prior to introducing the fluid warming device within a sterile package.

18. The fluid warming device of claim 17, wherein the battery is pre-charged and calibrated in accordance with a resistance of the heating element to include a charge sufficient to warm a pre-defined volume of fluid to a pre-defined temperature.

19. The fluid warming device of claim 11, further comprising a package including a casing, the casing defining a sterile interior region, the package enclosing and sealing the fluid warming device within the sterile interior region prior to use.

20. A sterile packaged medical device comprising:
a package including a casing, the casing defining a sterile interior region; and one of:
a fluid warming container enclosed within the sterile interior region of the casing, the fluid warming container including:
a base portion;
a sidewall portion coupled at a first end thereof to the base portion to define an interior volume of the fluid warming container, the interior volume arranged to receive a fluid;
a heating element;
a power source; and
a switch electrically connected to the power source and the heating element, the switch configured to selectively control a flow of electrical current from the power source to the heating element, whereby when the heating element receives the flow of electrical current, a temperature of the heating element increases; or
a fluid warming device comprising:
a housing having a first end, a second end, and a body extending therebetween;
a heating element;
a power source disposed within the housing; and
a switch electrically connected to the power source and the heating element, the switch configured to selectively control a flow of electrical current from the power source to the heating element, whereby when the heating element receives the flow of electrical current, a temperature of the heating element increases.
